(19) United States
(12) Patent Application Publication
(10) Pub. No.: US 2012/0004405 A1
(43) Pub. Date: Jan. 5, 2012

(54) COMPOUNDS HAVING ACTIVITY IN CORRECTING MUTANT CFTR CELLULAR PROCESSING

(76) Inventor: Peter Prehm, Nottuln (DE)

(21) Appl. No.: 13/139,243

(22) PCT Filed: Dec. 14, 2009
(86) PCT No.: PCT/EP2009/067124

§ 371 Date: Jun. 10, 2011

(30) Foreign Application Priority Data
Dec. 12, 2008 (EP) 08171521.1
Sep. 11, 2009 (EP) 09170073.2
Sep. 25, 2009 (EP) 09171378.4

(51) Int. Cl. C07H 7/02; C07C 233/54

(52) U.S. Cl. 536/53; 562/455

(57) ABSTRACT

The present invention relates to a compound which is characterized by the formula (I) or a pharmaceutically acceptable salt, solvate, hydrate thereof, wherein the ring systems A and B are independently selected from a monosaccharide, aryl (preferably phenyl), a heteroaryl or cycloalkyl (preferably cyclohexan), preferably with all substituents in equatorial configurations; R1 is independently selected from alkyl (preferably C1 to C6), a substituted or unsubstituted phenyl, preferably CH3; R2 is H, alkyl (preferably C1 to C6), a carbohydrate in a glycosidic β-linkage, preferably H; R3, R4, R5, and R6 are independently selected from H, (OH) hydroxy, alkyl preferably C1 to C6, alkoxy (preferably C1 to C6), amino, alkylamino (preferably C1 to C6), halogen, benzylamino, or benzoylamino; X is O, NH, alkylamino (NR), CO, S; and Y is O, NH, alkylamino (NR), CO, S. The present invention also relates to the compound of the invention and, optionally, a pharmaceutically acceptable carrier, for use in the treatment of (for treating) and/or preventing a disease or medical condition which is associated with mutant CFTR.

The present invention relates in general to a compound which is characterized by the formula

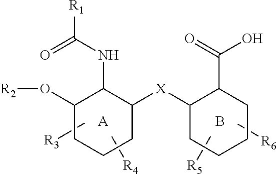

or a pharmaceutically acceptable salt thereof. The present invention further relates to pharmaceutical composition comprising the compound of the invention and to their use in the treatment of (for treating) and/or preventing diseases, disorders or medical conditions which are associated with mutant CFTR. The present invention also relates to a method for manufacturing a pharmaceutical composition comprising the steps of formulating the compound of the invention in a pharmaceutically acceptable form.

A variety of documents is cited throughout this specification. The disclosure content of said documents (including any manufacturer's specifications, instructions etc.) is herewith incorporated by reference; however, there is no admission that any document cited is indeed prior art as to the present invention.

The most common cause of cystic fibrosis (CF) is deletion of phenylalanine 508 (ΔF508) in the CF transmembrane conductance regulator (CFTR). The ΔF508 mutation produces defects in folding, stability, and channel gating. Cystic fibrosis (CF) is one of the most common inherited diseases, afflicting 1 in approximately 2,500 white individuals [1]. The primary cause of morbidity and mortality in CF is chronic lung infection and deterioration of lung function. CF is caused by mutations in the CF transmembrane conductance regulator (CFTR) gene, which encodes a cAMP-regulated chloride channel expressed at the apical membrane of epithelial cells in the airways, pancreas, testis, and other tissues [2;3]. The most common CFTR mutation producing CF is deletion of phenylalanine at residue 508 (ΔF508) in its amino acid sequence, which is present in at least 1 allele in approximately 90% of CF subjects [1]. The ΔF508-CFTR protein is misfolded and retained at the ER, where it is degraded rapidly [4-6]. The misfolding of ΔF508-CFTR is thought to be mild because it can be “rescued” in cell culture models by incubation for 18 hours or more at reduced (<30° C.) temperature (4) or with chemical chaperones such as glycerol [7] or phenylbutyrate [8], which results in partial restoration of ΔF508-CFTR plasma membrane expression. However, channel gating of the plasma membrane-rescued ΔF508-CFTR protein remains defective such that its open probability after cAMP stimulation is reduced by more than 3-fold compared with that of wild-type CFTR [9;10]. Small-molecule correctors of defective ΔF508-CFTR folding/cellular processing (“correctors”) and channel gating (“potentiators”) may provide a strategy for therapy of CF that corrects the underlying defect. A potential advantage of pharmacotherapy for defective ΔF508-CFTR processing and gating is that it minimizes concerns about treating the wrong cells or losing physiological CFTR regulation, as might occur with gene therapy or activation of alternative chloride channels. Recently, a number of small-molecule ΔF508-CFTR potentiators [11-13] and correctors have been identified [14-20]. These potentiators and correctors were mostly discovered by high-throughput screening for activation of the chloride channel.

The technical problem underlying the present invention is to provide means and methods for treating and/or preventing diseases or medical conditions which are associated with mutant CFTR.

The solution to this technical problem is achieved by providing the embodiments characterized in the claims.

It must be noted that as used herein, the singular forms “a”, “an”, and “the”, include plural references unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

We developed a new class of compounds that activated mutant-CFTR (potentiator function) and rescued it from intracellular degradation (corrector function). Functional correction was correlated with plasma membrane expression of the ΔF508-CFTR protein. These compounds may find use in the study and treatment of disorders related to mutant-CFTR, such as cystic fibrosis (“CF”) caused by the ΔF508 mutation. It is envisaged that the compounds of the present invention have mutant CFTR-corrector and/or mutant CFTR-potentiator function.

The invention also provides compositions and pharmaceutical preparations or compositions which comprise or consist of the novel compounds of the invention. The invention also features methods of use of such compounds or compositions in the treatment of a subject for disorders related to mutant-CFTR, such as cystic fibrosis, as well as kits and compound libraries useful for the study and treatment of disorders related to mutant-CFTR, such as cystic fibrosis.

A “mutant-CFTR” is the protein that results from a mutation, e.g., deletion mutation, insertion mutation, or point (substitution) mutation of the CFTR gene product relative to wildtype (e.g. ΔF508-CFTR, G551 D-CFTR, G1349D-CFTR, or D1 152H-CFTR). Said “mutant-CFTR” is further characterized as a dysfunctional CFTR as compared to a functional (e.g., wildtype) CFTR, where the dysfunction can encompass one or more of the following: (i) aberrant CFTR production like reduced CFTR production (e.g., at the level of transcription or translation); (ii) aberrant folding and/or trafficking (e.g. the mutant-CFTR is retained in the ER); (iii) abnormal regulation of conductance; (iv) decreases in chloride conductance (also called “gating defective mutant-CFTR”); (v); and the like. Said “mutant-CFTR” is encoded by a gene, or coding sequence, which encodes a mutant-CFTR.

One preferred example of a mutant-CFTR is ΔF508-CFTR. A “ΔF508-CFTR” is the protein that results from the deletion of a phenylalanine residue at amino acid position 508 of the CFTR gene product. A ΔF508-CFTR gene usually results from deletion of three nucleotides corresponding to the phenylalanine residue at amino acid position 508 of the encoded CFTR gene product. For an example of a gene that encodes ΔF508-CFTR, see, e.g. WO 91/102796.

A “disorder related to mutant-CFTR” means any medical condition, disorder or disease, or symptom of such condition, disorder, or disease that results from or is correlated with the presence of a mutant-CFTR (e.g., ΔF508-CFTR), e.g., chloride ion impermeability caused by reduced activity of ΔF508-CFTR in ion transport relative to a wild-type CFTR. Said term specifically includes cystic fibrosis (CF) which is sometimes also denoted as mucoviscidosis. A “disorder related to mutant-CFTR” encompasses conditions in an affected subject which are associated with the presence of a ΔF508-CFTR mutation on at least one allele, thus including subjects that carry a ΔF508-CFTR mutation on both alleles as well as heterozygous subjects having two different mutant forms of CFTR, e.g., a subject with one copy of ΔF508-CFTR and a copy of different mutant form of CFTR. Such different mutant forms (allelic variants), and a description of CF, including its symptoms, is found in Accession No. 602421 (entitled cystic fibrosis transmembrane conductance regulator; CFTR), and Accession No. 2 19700 (entitled Cystic fibrosis; CF) of the Online Mendelian Inheritance of Man database OMIM, as found at the world wide website of the National Institute of Health at ncbi.nlm.nih.gov. The terms “disorder”, “medical condition” and “disease” are used herein interchangeably.

As used herein and in the cystic fibrosis field a “potentiator” refers to a compound that increases the basal level (residual function) of ion transport by a mutant-CFTR (e.g. ΔF508-CFTR, G551 D-CFTR, G1349D-CFTR, or D1 152H-CFTR), where the mutant CFTR (in the absence of the compound) exhibits aberrantly low levels of ion transport relative to wildtype CFTR. As such, a “mutant-CFTR potentiator” refers to a potentiator compound that provides for an increased level of ion transport by a mutant-CFTR relative to ion transport capability of the mutant-CFTR in the absence of the compounds. It is therefore envisaged that the compounds of the present invention increase the ion transport rate, e.g. that of chloride ions, by a mutant-CFTR, preferably a mutant-CFTR (for example ΔF508-CFTR) that is comprised by a human epithelial cells (preferably epithelial cells of the respiratory tract), by about 5%, 10%, 20%, 30%, 40%, 50%, 60%, 70%, 80%, 90% or even 100% or more, when compared to the transport rate that is achieved without the addition of said compound. It is preferred that said mutant-CFTR is ΔF508-CFTR. A test which enables the skilled person to screen or test for a potentiator is the well-known iodide efflux technique which is exemplarily set out in Example 4.

As used herein and in the cystic fibrosis field a “corrector” is a compound that increases the level of ion transport by a mutant-CFTR relative to ion transport in the absence of the compound by correcting the underlying defect of the CFTR polypeptide, e.g., a defect that results from post-translational mis-processing (e.g., misfolding). In contrast to the “potentiator”, which merely increases the residual function of the mutant-CFTR, correctors take corrective action on the underlying effect, which is causative for the reduced ion transport mediated by CFTR (e.g. at the level of transcription or translation; aberrant folding and/or trafficking etc). CFTR correctors of the invention of particular interest are those that facilitate correction of specific mutant-CFTRs, preferably ΔF508-CFTR. Mutant-CFTR correctors are usually exhibit high affinity for one or more mutant-CFTRs, e.g., have an affinity for mutant-CFTR of at least about one micromolar, about one to five micromolar, about 200 nanomolar to one micromolar, about 50 nanomolar to 200 nanomolar, or below 50 nanomolar. Correctors may facilitate posttranslational folding of newly synthesized ΔF508-CFTR and/or enhance the stability of mature ΔF508-CFTR.

As used herein, a “mutant-CFTR corrector-potentiator” is a compound that exhibits both mutant-CFTR corrector and potentiator activity, or a plurality of compounds comprising compounds that exhibit corrector function and compounds that exhibit potentiator function. This compound/these compounds usually exhibit high affinity for one or more mutant-CFTRs, e.g., have an affinity for mutant-CFTR of at least about one micromolar, about one to five micromolar, about 200 nanomolar to one micromolar, about 50 nanomolar to 200 nanomolar, or below 50 nanomolar.

The lead structure for the design of the compounds of the present invention is depicted below:

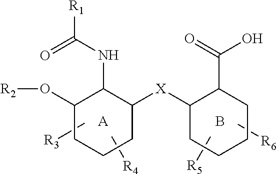

the compounds of the present invention obey, preferably, the rule of 5 for “drugable” compounds, i.e.:
there are not more than 5H-bond donors (sum of OH and NH) in the molecule;

there are no more than 10H-bond acceptors (sum of N and O) in the molecule;

the molecular weight does not exceed 500;

log P does not exceed 5; and
the PSA (Molecular polar surface area) does not exceed 150.

These features can conveniently be calculated by the skilled person, for example when using the information contained in the free website http://www.molinspiration.com/cqi-bin/properties. However, even without the information provided by hr referenced webpage, the skilled person is in a position to design a compound which obeys the above stated well-recognized rules of 5 for “drugable” compounds.

The present invention, thus, relates to a compound which is characterized by the following formula

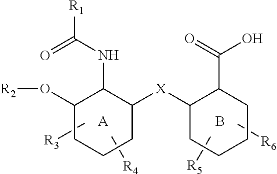

or a pharmaceutically acceptable derivative thereof (e.g. a pharmaceutically acceptable salt, hydrate, solvate, stereoisomer and/or prodrug), wherein
the ring systems A and B are independently selected from a monosaccharide, aryl (preferably phenyl), a heteroaryl or cycloalkyl (preferably cyclohexan), or pyran, preferably with all substituents in equatorial configurations;
R1 is selected from H, alkyl (preferably C1 to C6), a substituted or unsubstituted phenyl, preferably CH3;
R2 is H, alkyl (preferably C1 to C6), a carbohydrate in a glycosidic β-linkage, preferably H;
R3, R4, R5, and R6 are independently selected from H, (OH) hydroxy, alkyl preferably C1 to C6, alkoxy (preferably C1 to C6), amino, alkylamino (preferably C1 to C6), halogen, benzylamino, benzoylamino and/or alkanolyl (preferably C1 to C6; hydroxymethyl or hydroxylethyl being more preferred); it is also envisaged that the ring system A and/or B comprises additional substituents besides the mentioned R3,
R4, R5, and R6—these additional substituents are likewise independently selected from H, (OH) hydroxy, alkyl preferably C1 to C6, alkoxy (preferably C1 to C6), amino, alkylamino (preferably C1 to C6), halogen, benzylamino, benzoylamino and/or alkanolyl (preferably C1 to C6; hydroxymethyl or hydroxylethyl being more preferred);
X is O, NH, alkylamino (NR), CO, S; and
Y is O, NH, alkylamino (NR), CO, S.

It is envisaged that R1, R2, R3, R4, R5 and/or R6 are either substituted (for example halogenated, preferably with chloride) or unsubstituted.

It is envisaged that the compounds of the present invention have mutant-CFTR corrector and/or mutant CFTR-potentiator function. It is preferred that said mutant-CFTR is ΔF508-CFTR.

Furthermore, it has to be understood that the compounds of the present invention, can be further modified to achieve (i) modified organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmacokinetic parameters (resorption, distribution, metabolism and excretion), and/or (vii) modified physico-chemical parameters (solubility, hygroscopicity, color, taste, odor, stability, state).

It is, for example, envisaged that the carboxyl group in ring B of the depicted formulas can be masked as an ester to prevent serious side effects due to stomach ulceration, a well known phenomenon for acidic nonsteroidal antirheumatic drugs (NSARD). These esters are readily cleaved by serum or cytosolic esterases to form the active acidic compound. The alcohol that forms the ester can carry additional functional groups such in nitric oxide releasing aspirin derivatives [260].

The term “pharmaceutically acceptable derivatives” of a compound of the invention include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

The term “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The term “pharmaceutically acceptable ester” of a compound of the invention means an ester that is pharmaceutically acceptable and that possesses the desired pharmacological activity of the parent compound, and includes, but is not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. The term “pharmaceutically acceptable enol ether” of a compound of the invention means an enol ether that is pharmaceutically acceptable and that possesses the desired pharmacological activity of the parent compound, and includes, but is not limited to, derivatives of formula C═C(OR) where R is hydrogen, alkyl, alkenyl, alkynyl, aryl, heteroaryl, aralkyl, heteroaralkyl, cycloalkyl or heterocyclyl. The term “pharmaceutically acceptable enol ester” of a compound of the invention means an enol ester that is pharmaceutically acceptable and that possesses the desired pharmacological activity of the parent compound, and includes, but is not limited to, derivatives of

formula C═C(OC(O)R) where R is hydrogen, alkyl, alkenyl, alkynyl, aryl, heteroaryl, aralkyl, heteroaralkyl, cycloalkyl or heterocyclyl.

The term “pharmaceutically acceptable solvate or hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

The term “benzylamino” refers to an amino group substitute with an benzyl group.

The term “benzoylamino” refers to an amino group substitute with an benzoyl group.

The terms “alkyl” and “alkylene” as used herein, whether used alone or as part of another group, refer to substituted or unsubstituted aliphatic hydrocarbon chains, the difference being that alkyl groups are monovalent (i.e., terminal) in nature whereas alkylene groups are divalent and typically serve as linkers. Both include, but are not limited to, straight and branched chains containing from 1 to about 12 carbon atoms, preferably 1 to about 6 carbon atoms, unless explicitly specified otherwise. For example, methyl, ethyl, propyl, isopropyl, butyl, i-butyl and t-butyl are encompassed by the term “alkyl.” Specifically included within the definition of “alkyl” are those aliphatic hydrocarbon chains that are optionally substituted. Representative optional substituents include, but are not limited to, hydroxy, oxo (═O), acyloxy, alkoxy, amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl. Preferred substituents include halogens, —CN, —OH, oxo (═O), and amino groups.

The carbon number as used in the definitions herein refers to carbon backbone and carbon branching, but does not include carbon atoms of the substituents, such as alkoxy substitutions and the like.

The term “alkenyl”, as used herein, whether used alone or as part of another group, refers to a substituted or unsubstituted aliphatic hydrocarbon chain and includes, but is not limited to, straight and branched chains having 2 to about 10 carbon atoms (unless explicitly specified otherwise) and containing at least one double bond. Preferably, the alkenyl moiety has 1 or 2 double bonds. Such alkenyl moieties can exist in the E or Z conformations and the compounds of this invention include both conformations. Specifically included within the definition of “alkenyl” are those aliphatic hydrocarbon chains that are optionally substituted. Representative optional substituents include, but are not limited to, hydroxy, acyloxy, alkoxy, amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl. Heteroatoms, such as O or S attached to an alkenyl should not be attached to a carbon atom that is bonded to a double bond. Preferred substituents include halogens, —CN, —OH, and amino groups

The term “alkynyl”, as used herein, whether used alone or as part of another group, refers to a substituted or unsubstituted aliphatic hydrocarbon chain and includes, but is not limited to, straight and branched chains having 2 to about 10 carbon atoms (unless explicitly 0 specified otherwise) and containing at least one triple bond. Preferably, the alkynyl moiety has about 2 to about 7 carbon atoms. In certain embodiments, the alkynyl can contain more than one triple bond and, in such cases, the alkynyl group must contain at least three carbon atoms. Specifically included within the definition of “alkynyl” are those aliphatic hydrocarbon chains that are optionally substituted. Representative optional substituents include, but are not limited to, hydroxy, \acyloxy, alkoxy, amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl. Preferred substituents include halogens, —CN, —OH, and amino groups Heteroatoms, such as O or S attached to an alkynyl should not be attached to the carbon that is bonded to a triple bond.

The term “cycloalkyl” as used herein, whether alone or as part of another group, refers to a substituted or unsubstituted alicyclic hydrocarbon group having 4 to about 7 carbon atoms, with 5 or 6 carbon atoms being preferred. “Cyclohexane” is even more preferred.

Specifically included within the definition of “cycloalkyl” are those alicyclic hydrocarbon groups that are optionally substituted. Representative optional substituents include, but are not limited to, hydroxy, oxo (═O), acyloxy, alkoxy, amino, amino substituted by one or two alkyl groups of from 1 to 6 carbon atoms, aminoacyl, acylamino, thioalkoxy of from 1 to 6 carbon atoms, substituted thioalkoxy of from 1 to 6 carbon atoms, and trihalomethyl.

The term “aryl”, as used herein, whether used alone or as part of another group, is defined as a substituted or unsubstituted aromatic hydrocarbon ring group having 5 to about 10 carbon atoms (unless explicitly specified otherwise) with 5 to 7 carbon atoms being preferred. The “aryl” group can have a single ring or multiple condensed rings. The term“aryl” includes, but is not limited to phenyl, a-naphthyl, (3-naphthyl, biphenyl, anthryl, tetrahydronaphthyl, fluorenyl, indanyl, biphenylenyl, and acenaphthenyl. “Phenyl” is even more preferred.

Specifically included within the definition of “aryl” are those aromatic groups that are optionally substituted. In representative embodiments of the present invention, the, “aryl” groups are optionally substituted with from 1 to 5 substituents selected from the group consisting of acyloxy, hydroxy, acyl, alkyl of 1 to 6 carbon atoms, alkoxy of 1 to 6 carbon atoms, alkenyl of 2 to 6 carbon atoms, alkynyl of 2 to 6 carbon atoms, substituted alkyl, substituted alkoxy, substituted alkenyl, substituted alkynyl, amino, amino substituted by one or two alkyl groups of from 1 to 6 carbon atoms, aminoacyl, acylamino, azido, cyano, halo, nitro, thioalkoxy of from 1 to 6 carbon atoms, substituted thioalkoxy of from 1 to 6 carbon atoms, and trihalomethyl. For example, the“aryl” groups can be optionally substituted with from 1 to 3 groups selected from Cl-C6 alkyl, Cl-C6 alkoxy, hydroxy, C3-C6 cycloalkyl, —(CH2)-C3-C6 cycloalkyl, halogen, Cl-C3 perfluoroalkyl, Cl-C3 perfluoroalkoxy, —(CH2) q-phenyl, and —O(CH2) q-phenyl. In these embodiments, the phenyl group of —(CH2) q-phenyl and —O(CH2) q-phenyl can be optionally substituted with from 1 to 3 groups selected from Cl-C6 alkyl, Cl-C6 alkoxy, phenyl, halogen, trifluoromethyl or trifluoromethoxy. In other embodiments, phenyl groups of the present invention are optionally substituted with from 1 to 3 groups selected from C1-C6 alkyl, C1-C6 alkoxy, —(CH2) p-phenyl, halogen, trifluoromethyl or trifluoromethoxy. Preferred aryl groups include phenyl and naphthyl. Preferred substituents on the aryl groups herein include alkyl, alkoxy, halo, cyano, nitro, trihalomethyl, and thioalkoxy

As used herein, the term “heteroaryl”, whether used alone or as part of another group, is defined as a substituted or unsubstituted aromatic heterocyclic ring system (monocyclic or bicyclic). Heteroaryl groups can have, for example, from about 3 to about 50 carbon atoms (unless explicitly specified otherwise), with from about 4 about 10 being preferred. In some embodiments, heteroaryl groups are aromatic heterocyclic ring systems having about 4 to about 14 ring atoms and containing carbon atoms and 1,2, or 3 oxygen, nitrogen or sulfur heteroatoms. Representative heteroaryl groups are furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Specifically included within the definition of “heteroaryl” are those aromatic heterocyclic rings that are substituted with 1 to 5 substituents selected from the group consisting of acyloxy, hydroxy, acyl, alkyl of 1 to 6 carbon atoms, alkoxy of 1 to 6 carbon atoms, alkenyl of 2 to 6 carbon atoms, alkynyl of 2 to 6 carbon atoms, substituted alkyl, substituted alkoxy, substituted alkenyl, substituted alkynyl, amino, amino substituted by one or two alkyl groups of from 1 to 6 carbon atoms, aminoacyl, acylamino, azido, cyano, halo, nitro, thioalkoxy of from 1 to 6 carbon atoms, substituted thioalkoxy of from 1 to 6 carbon atoms, and trihalomethyl. In some embodiments of the present invention, the “heteroaryl” groups can be optionally substituted with from 1 to 3 groups selected from Cl-C6 alkyl, Cl-C6 alkoxy, hydroxy, C3-C6 cycloalkyl, —(CH2)-C3-C6 cycloalkyl, halogen, Cl-C3 perfluoroalkyl, C1-C3 perfluoroalkoxy, —(CH2) q-phenyl, and —O(CH2) q-phenyl. In these embodiments, the phenyl group of —(CH2) q-phenyl and —O(CH2) q-phenyl can be optionally substituted with from 1 to 3 groups selected from Cl-C6 alkyl, Cl-C6 alkoxy, phenyl, halogen, trifluoromethyl or trifluoromethoxy. Preferred heterocycles of the present invention include substituted and unsubstituted furanyl, thiophenyl, benzofuranyl, benzothiophenyl, indolyl, pyrazolyl, oxazolyl, and fluorenyl.

As used herein, the term “phenylcycloalkyl”, whether used alone or as part of another group, refers to the group Ra-Rb-wherein Rb is an optionally substituted cyclized alkyl group having from about 3 to about 10 carbon atoms with from about 3 to about 6 being preferred and Ra is an optionally substituted phenyl group as described above. Preferred cycloalkyl groups are cyclopropyl, cyclobutyl, cyclopentyl or cyclohexyl. Examples of phenylcycloalkyl also include groups of formula: EMI9.1 wherein R7 and R8 are, independently, hydrogen, Cl-C6 alkyl, Cl-C6 alkoxy, hydroxy, —(CH2) q-phenyl, —O(CH2) q-phenyl, C3-C6 cycloalkyl, halogen, Cl-C3 perfluoroalkyl or Cl-C3 perfluoroalkoxy; m is from 1 to 4, and q=0-6.

The term “alkoxy” as used herein, refers to the group RA-O-wherein Ra is an alkyl group as defined above. Specifically included within the definition of “alkoxy” are those alkoxy groups that are optionally substituted. Preferred substituents on alkoxy and thioalkoxy groups include halogens, —CN, —OH, and amino groups

The term “arylalkyl” or “aralkyl” refers to the group-Ra-Rb, where Ra is an alkyl group as defined above, substituted by Rb, an aryl group, as defined above. Aralkyl groups of the present invention are optionally substituted. Examples of arylalkyl moieties include, but are not limited to, benzyl, 1-phenylethyl, 2-phenylethyl, 3-phenylpropyl, 2-phenylpropyl and the like.

The term “halogen” or “halo” refers to chlorine, bromine, fluorine, and iodine.

The term “alkylamino” refers to groups having the formula selected from: (a) —(CH2)m-NH2, where m=1 to 10, (b) —NH—(CH2)n-NH2, where n=1 to 10, or (c) —NH—(C2H4NH)xC2H4NH2, where x=0 to 5.

The term “monosaccharide” includes trioses like glyceraldehyde or dihydroxyacetone; tetroses like erythrose, threose or erythrulose; pentoses like arabinose, lyxose, ribose, deoxyribose, xylose, ribulose and xylulose; hexoses like allose, altrose, galactose, glucose, gulose, idose, mannose, fructose, psicose, sorbose tagatose and talose; heptoses like mannoheptulose, sedoheptulose; octoses like octolose, 2-keto-3-deoxy-manno-octonate or nonoses like sialose.

The term “carbohydrate” includes monosaccharides as defined above, disaccharides, or oligosaccharides consisting of 1 to 10, preferably 1 to 3 monosaccharides.

It is preferred that the compounds of the invention are membrane-permeable. “Membrane-permeable” means that the compounds of the invention are able to enter a mammalian cell, preferably a human cell and even more preferred a human epithelial cell, epithelial cells of the respiratory tract being most preferred. Examples of human epithelial cell lines include A549, HPL1, or Calu-3.

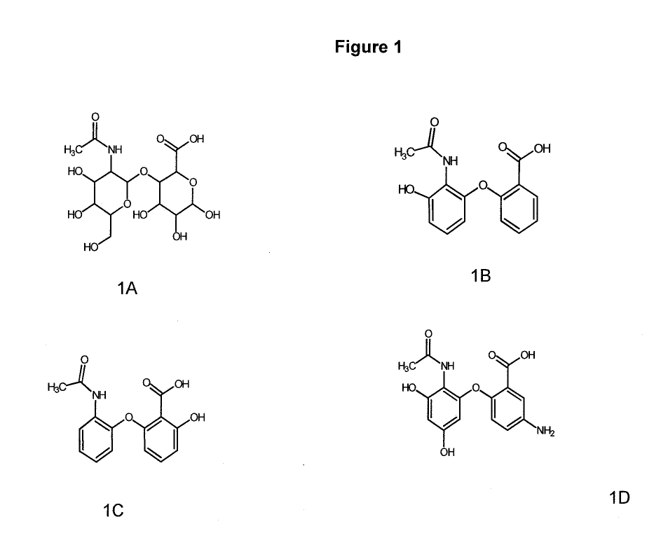

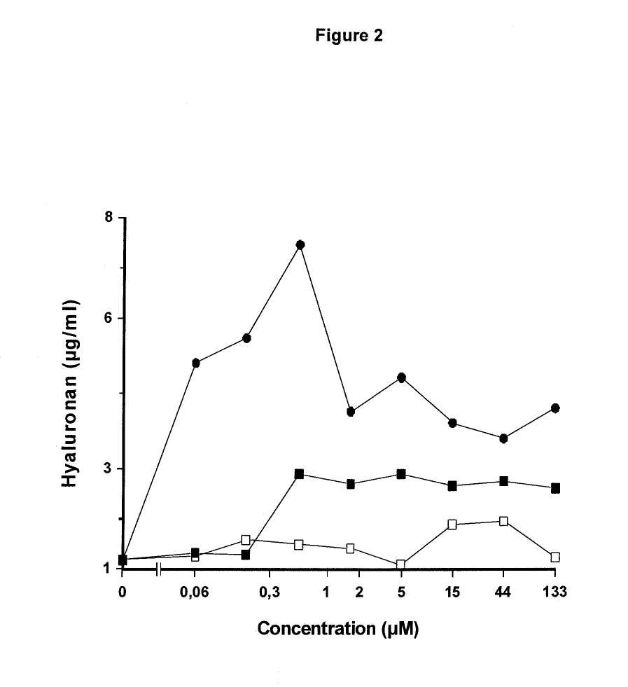

We synthesized the diaryl analogs of hyaluronan disaccharides (FIG. 1A): 2-(2-acetamido-3-hydroxyphenoxy)benzoic acid (FIG. 1B) and 2-(2-acetamidophenoxy)-6-hydroxybenzoic acid (FIG. 1C). The structures differ only in the position of one hydroxyl group being in o-position of the acetylamido group in compound 1B or in o-position of the carboxyl group in 1C. Thus these compounds resemble the non-reducing end of a hyaluronan chain with a terminal N-acetylamino group for 1B and with a terminal glucuronic acid for 1C. Some of these compounds were tested initially for their effect on hyaluronan export from human fibroblasts. To much of our surprise and contrary to our expectation, they were activating, i.e. they increased the hyaluronan export from human fibroblasts. We modified compound 1B by introducing additional hydroxyl, amino, or hydrophobic groups. All these compounds were also activating and the most active one was 2-(2-acetamido-3,5-dihydroxyphenoxy)-5-aminobenzoic acid (FIG. 1D).

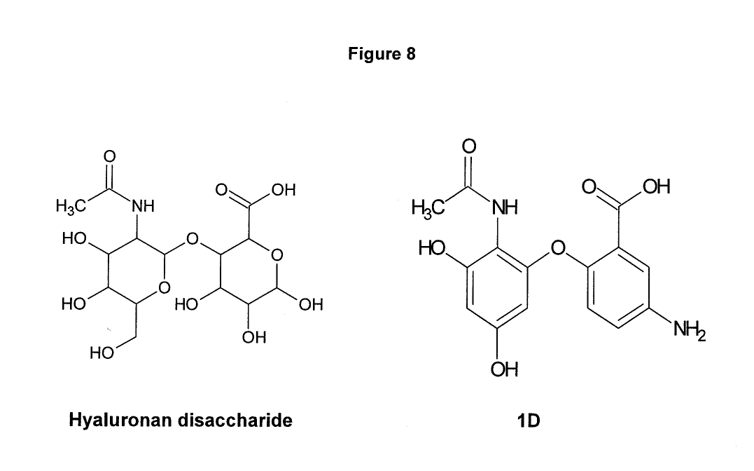

Thus, in its broadest sense, the present invention relates to diaryl analogs of the hyaluronan dissacharide (the hyaluronan dissacharide is depicted in FIG. 8), which increase the hyaluronan export from a human cell (preferably fibroblasts), preferably about 5%, 10%, 20%, 30%, 40%, 50%, 60%, 70%, 80%, 90% or even 100% or more, when compared to the transport rate that is achieved without the addition of said compound. Such compounds are structurally exemplified herein.

In view of the above, it is envisaged that the compounds of the present invention increase (and thereby activate) the hyaluronan export from a human cell (preferably fibroblasts), preferably about 5%, 10%, 20%, 30%, 40%, 50%, 60%, 70%, 80%, 90% or even 100% or more, when compared to the transport rate that is achieved without the addition of said compound. One assay for determining the hyaluronan export is exemplified in Example 3, i.e. it is envisaged that the hyaluronan export is activated about 5%, 10%, 20%, 30%, 40%, 50%, 60%, 70%, 80%, 90% or even 100% or more as exemplified above in an experimental setting as specified in Example 3. Another specific screening assay for the hyaluronan transporter is based on the extrusion of labelled hyaluronan oligosaccharides from intact cells in monolayer culture. Said assay is further explained in WO2005/013947, particularly in the appended examples of said document (e.g Example 8 or Example 11). In such cases it is sufficient to analyse the effect of the activator e.g. on a cell comprising CFTR, i.e. one compares the hyaluronan-transport before and after the addition of the activator and thereby identifies compounds which increase the transport-rate of hyaluronan across a lipid bilayer.

In a preferred embodiment the compounds of the present invention specifically increase(s) the transport of hyaluronan across a lipid bilayer mediated by CFTR. The term “specifically increase(s)” used in accordance with the present invention means that the compound specifically causes an increase of the transport of hyaluronan as mediated by CFTR but has no or essentially has no significant effect on other cellular proteins or enzymes.

The present invention also relates to a screening method for the screening of compounds disposed to (a) prevent the onset of cystic fibrosis (CF); (b) to ameliorate the symptoms of CF; (c) to treat CF, or (d) to facilitate posttranslational folding of ΔF508-CFTR and/or to enhance the stability of ΔF508-CFTR; said method comprising the step:

(a) analyzing the capability of said compound to increase (preferably specifically as defined herein above) the hyaluronan export from a human cell (preferably a fibroblast; more preferably a MRP5 deficient cell, and even more preferably a MRP5 deficient but CFTR positive cell), wherein said compound is disposed to (a) prevent the onset of cystic fibrosis (CF); (b) to ameliorate the symptoms of CF; (c) to treat CF, or (d) to facilitate posttranslational folding of ΔF508-CFTR and/or to enhance the stability of ΔF508-CFTR, if it has the capability to increase said hyaluronan export. Means and methods to put this method into practice are disclosed herein.

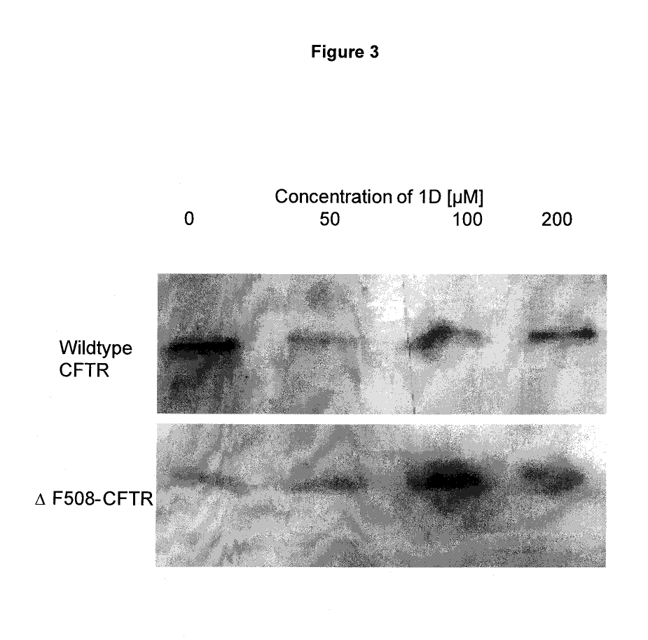

The ΔF508-CFTR mutation impairs conformational maturation and transport competence at the endoplasmic reticulum and destabilizes ΔF508-CFTR in post-Golgi compartments. Correctors may facilitate posttranslational folding of newly synthesized ΔF508-CFTR and/or enhance the stability of mature ΔF508-CFTR. Therefore we analysed the CFTR expression on the cell surface in the presence of increasing concentration of compound 1D on human epithelial cells containing wildtype and ΔF508-CFTR by Western blotting with anti-CFTR. FIG. 3 shows that the expression of wildtype CFTR was slightly decreased by 1D, whereas the expression of ΔF508-CFTR was enhanced. This result indicates that compound 1D enhanced cellular processing of ΔF508-CFTR, i.e. that compound 1D has corrector function.

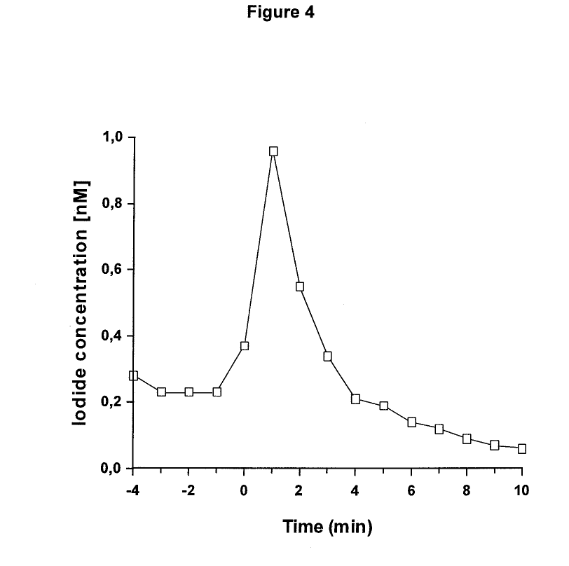

We than used the iodide efflux technique to assess the effect of compound 1D on ΔF508-CFTR epithelial cells. FIG. 4 shows that compound 1D stimulated a sudden burst of iodide efflux from the cells. The immediate opening of the channels indicates that compound 1D also functions as a potentiator.

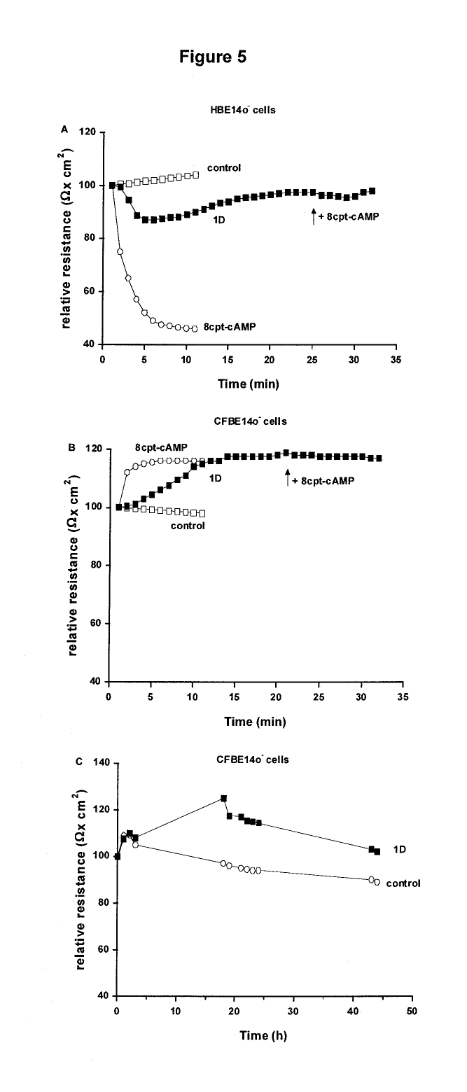

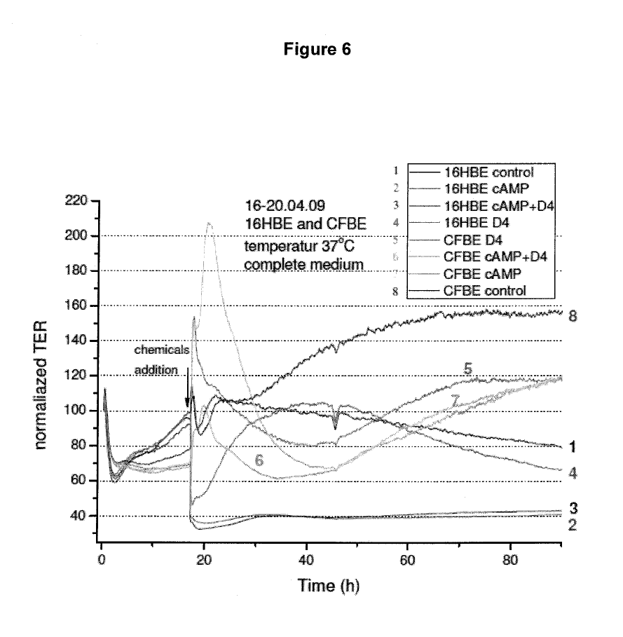

The transport activity of epithelial cells can conveniently be measured by the transepithelial resistance. We measured the kinetics of the relative resistance of wildtype HBE14o- and mutant CFBE14o- in the presence of compound 1D or the membrane permeable cAMP analogue 8 cpt-cAMP that activates CFTR. The response to elevated intracellular cAMP levels differs markedly between wildtype and mutant cells (FIG. 5). Compound 1D at 10 μM concentration had similar effects as 8 cpt-cAMP on wildtype as well as mutant cells (FIGS. 5A and 5B), because subsequent addition of 8 cpt-cAMP did not cause any further change indicating that it opened the CFTR channels. Since we observed an increase of membrane expression on ΔF508-CFTR epithelial cells with compound 1D only at 100 μM concentrations, we also measured the transepithelial resistance at 100 μM over a longer time period (FIG. 5C). Again we observed an increase in transepithelial resistance which hat its maximum between 5 to 18 hours, indicating that 1D also rescued on ΔF508-CFTR from intracellular degradation in addition to direct activation. FIG. 6 shows a detailed simultaneous analysis of the long term effects both on wild type and ΔF508-CFTR epithelial cells and its comparison to the activator 8 cpt-cAMP.

Recently, we discovered that CFTR can export the extracellular polysaccharide hyaluronan in addition to chloride and that this export is defective in patients with cystic fibrosis leading to highly viscous mucous of aggregated hyaluronan protein mixtures. This finding led to the concept that membrane permeable hyaluronan analogs might alter hyaluronan and/or chloride export. We synthesized two disaccharide analogs that differed only in the position of one hydroxyl group mimicking either the non-reducing terminus GlcNac or GlcA. These compounds were first tested on human fibroblasts cultures for their influence on hyaluronan exporter MRP5. Surprisingly, the disaccharide with the non-reducing terminus GlcNac was activating, whereas the non-reducing terminus GlcA was inactive. Further modifying the chemical structure of the activating dissacharide led to the hitherto most activating compound 1D. Compound 1D also activated hyaluronan export through CFTR in a mouse fibroblasts cell line. Since epithelial cell lines that export hyaluronan through CFTR are not available we analysed the effect of compound 1D on chloride transport activity of wildtype and mutant epithelial cell lines. It corrected ΔF508-CFTR cellular misprocessing and restored plasma membrane expression and halide permeability. We verified correction by electrophysiological and biochemical measurements. In wildtype cells it opens CFTR channels and intracellular chloride is exported reducing the resistance. In mutants cells it also opens the channels. But due to the altered transepithelial potential, chloride is imported into the cytosol, where it inhibits the import of Na+ by ENac [27;28].

The identification of small-molecule ΔF508-CFTR correctors presented a greater conceptual difficulty than that of ΔF508-CFTR potentiators or CFTR activators/inhibitors because correction of cellular misprocessing could involve multiple targets, whereas the primary target for potentiators, activators, and inhibitors is CFTR itself. CFTR cellular processing involves translation, folding at the ER, Golgi transport, posttranslational glycosylation, and apical plasma membrane targeting [29]. Plasma membrane CFTR is internalized by endocytosis and then recycled to the plasma membrane or targeted for lysosomal degradation [30]. ΔF508-CFTR folding is inefficient, with 99.5% of newly synthesized ΔF508-CFTR in BHK cells targeted for degradation without reaching the Golgi apparatus. Our results thus provide proof-of-principle for discovery of small-molecule correctors of ΔF508-CFTR cellular misprocessing. It has been estimated that 6-10% of normal CFTR activity might prevent or significantly reduce lung pathology in CF [31].

In a preferred embodiment, the present invention relates to compounds 1B, 1C, 1D 1F and 1G. The toxicity of compound 1D was measured by the Alamarblue® assay (Invitrogen) up to concentrations of 400 μM, and it was found to be not toxic (data not shown). Compound 1D is particularly preferred.

The formulas of said compounds are depicted in the table below.

| | |
|---|---|
| 1B | 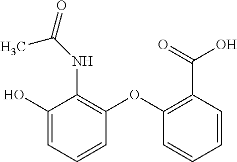 |
| 1C | 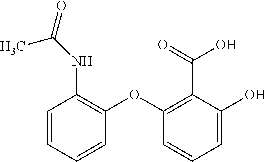 |
| 1D | 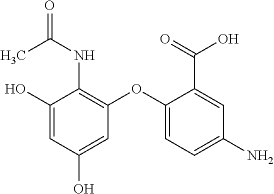 |
| 1F (d408) | 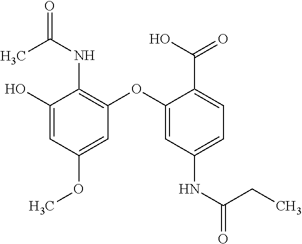 |
| 1G (amin 30) | 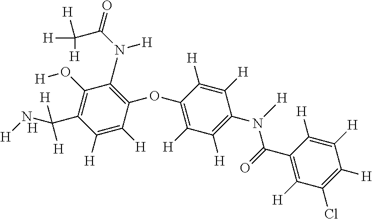 |

The present invention also relates to a compound based on compounds 1B, 1C, 1D, 1F and/or 1G. “Based on” means chemically altered derivatives, which derivatives have, preferably, a comparable biological function when compared with one of the compounds selected from, 1B, 1C, 1D, 1F and/or 1G, 1D being preferred. “Comparable biological function” means that the chemical derivatives of the invention are still able to act as potentiator and/or correctors with a deviation of the potentiator and/or corrector activity in respect to one of the compounds selected from, 1B, 1C, 1D, 1F and/or 1G, 1D being preferred, of not more than about 40%, 30%, 20%, 15%, 10%, 5%, 2.5%, 2% or 1%, for example under conditions which equate to or are identical with those set out in the respective Examples.

The compounds of the invention may be employed for the preparation of a pharmaceutical composition for treating and/or preventing diseases or medical conditions which are associated with mutant CFTR. Such diseases/medical conditions are explained herein elsewhere.

Other activators which increase the hyaluronan transport rate are exemplified and described in PCT/EP2009/067119.

The pharmaceutical composition of the present invention may optionally comprise a pharmaceutical carrier.

Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use.

Upon using the compounds of the present invention, it is possible to treat/ameliorate and/or prevent diseases or medical conditions which are associated with mutant CFTR. It is thus envisaged that the compounds of the present invention are used for the preparation of a pharmaceutical composition for the treatment of diseases or medical conditions which are associated with mutant CFTR, preferably for the treatment of cystic fibrosis.

The skilled person is well aware which specific diseases are associated with mutant CFTR and, provided with the teaching and disclosure of the present invention can easily test for such a mutant CFTR. Thus, it is possible to identify a subject at risk for a disease which is associated with mutant CFTR or to diagnose a disease which is associated with mutant CFTR. This can be diagnosed e.g., by isolating cells from an individual. Such cells can be collected from body fluids, skin, hair, biopsies and other sources.

The compounds of the present invention are therefore useful/may therefore be used for the medical treatment of cystic fibrosis.

It has to be understood that in the context of the present invention, “a compound of the invention” includes “at least one compound of the invention”, wherein the term “at least one” comprises at least one, at least two, at least three, at least four, at least five, at least six . . . etc. compound(s) of the invention. It will be understood that the number of compounds which are used together (simultaneously or displaced) will be selected on a case to case basis in order to provide a suitable treatment for the cell/tissue/subject. In this context, “suitable” means that the treatment with the respective activator(s) of the invention exerts a beneficial effect, e.g. it prevents, counters or arrests the progress of the condition.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

The compounds of the present invention can be applied prophylactically.

Thus in a further embodiment of the medical uses of the present invention said compounds(s) is(are) to be administered prophylactically.

Alternatively, the compounds can by applied therapeutically, preferably as early as possible.

Thus, in another embodiment of the medical uses of the present invention said compound(s) is(are) to be administered therapeutically.

The dosage regimen utilizing the compounds of the present invention is selected in accordance with a variety of factors including type, species, age, weight, sex and medical condition of the patient; the severity of the condition to be treated; the route of administration; and the particular compound employed. It will be acknowledged that an ordinarily skilled physician or veterinarian can easily determine and prescribe the effective amount of the compound required to prevent, counter or arrest the progress of the condition.

It is also envisaged that the compounds of the present invention are employed in co-therapy approaches, i.e. in co-administration with other medicaments or drugs.

The present invention also relates to a method of preventing, ameliorating and/or treating the symptoms of a disease or medical conditions which is associated with mutant CFTR in a subject, comprising administering at least one compound/composition as defined herein to the subject.

In the context of the present invention the term “subject” means an individual in need of a treatment of an affective disorder. Preferably, the subject is a mammalian, particularly preferred a human, a horse, a camel, a dog, a cat, a pig, a cow, a goat or a fowl.

The concentration of therapeutically active compound in the formulation may vary from about 0.1-100 wt %. The administration of the compounds and/or pharmaceutical composition of the invention can be done in a variety of ways as discussed above, including, but not limited to, orally, subcutaneously, intravenously, intra-arterial, intranodal, intramedullary, intrathecal, intraventricular, intranasally, intrabronchial, transdermally, intranodally, intrarectally, intraperitoneally, intramuscularly, intrapulmonary, vaginally, rectally, or intraocularly. Preferred is intrapulmonary. In some instances the compounds and/or compositions may be directly applied as a solution spray or with an inhaler.

Drugs or pro-drugs after their in vivo administration are metabolized in order to be eliminated either by excretion or by metabolism to one or more active or inactive metabolites (Meyer, J. Pharmacokinet. Biopharm. 24 (1996), 449-459). Thus, rather than using the actual compound as defined herein, a corresponding formulation as a pro-drug can be used which is converted into its active in the patient. Precautionary measures that may be taken for the application of pro-drugs and drugs are described in the literature; see, for review, Ozama, J. Toxicol. Sci. 21 (1996), 323-329.

This disclosure may best be understood in conjunction with the accompanying drawings, incorporated herein by references. Furthermore, a better understanding of the present invention and of its many advantages will be had from the following examples, given by way of illustration and are not intended as limiting.

The figures show:

FIG. 1 Structures of hyaluronan disaccharide and analogs: 1A, Hyaluronan disaccharide; 1B, 2-(2-acetamido-3-hydroxyphenoxy)benzoic acid; 1C, 2-(2-acetamidophenoxy)-6-hydroxybenzoic acid; 1D, 2-(2-acetamido-3,5-dihydroxyphenoxy)-5-aminobenzoic acid.

FIG. 2 Activation of hyaluronan export from human fibroblasts by hyaluronan disaccharide analogs. Fibroblasts were grown to 50% confluency and incubated for two days with the hyaluronan analogs (▪) 1B; (□) 1C; () 1D in increasing concentrations. Hyaluronan was determined in the culture supernantant.

FIG. 3 Surface expression of CFTR in human epithelial cells. Human epithelial cells containing wildtype (A) or ΔF508-CFTR (B) were incubated with increasing concentrations of compound 1D and the amount of CFTR on the cell surface was analysed by Western blotting.

FIG. 4 Compound 1B pretreatment stimulates iodide efflux from ΔF508-CFTR epithelial cells. Data show the time course of iodide efflux from ΔF508-CFTR epithelial cells. Cells loaded with iodide were treated with compound 1D at the time point 0. The extracellular iodide concentration was determined as described in the Methods.

FIG. 5 Transepithelial resistance (TER)— The transepithelial resistance was determined in wildtype HBE14o- (A) and mutant CFBE14o- in the absence (□) or presence of to the membrane permeable CFTR activator 8 cpt-cAMP (8-(4-Chlorophenylthio)-adenosine-3′,5′-cyclic monophosphate) at 100 μM concentration (∘) or 10 μM of compound 1D (▪) added at time point 0, when the relative resistance was set to 100. The culture containing compound 1D was supplemented with 8 cpt-cAMP at the times indicated. The result shows that compound 1D is agonistic to 8 cpt-cAMP. At a 100 μM concentration, compound 1D led to long lasting rescue of ΔF508-CFTR (C).

FIG. 6 FIG. 6 shows a detailed simultaneous analysis of the long term effects both on wild type and ΔF508-CFTR epithelial cells and its comparison to the activator 8 cpt-cAMP.—“D4” depicted in that figure is compound 1D as defined herein

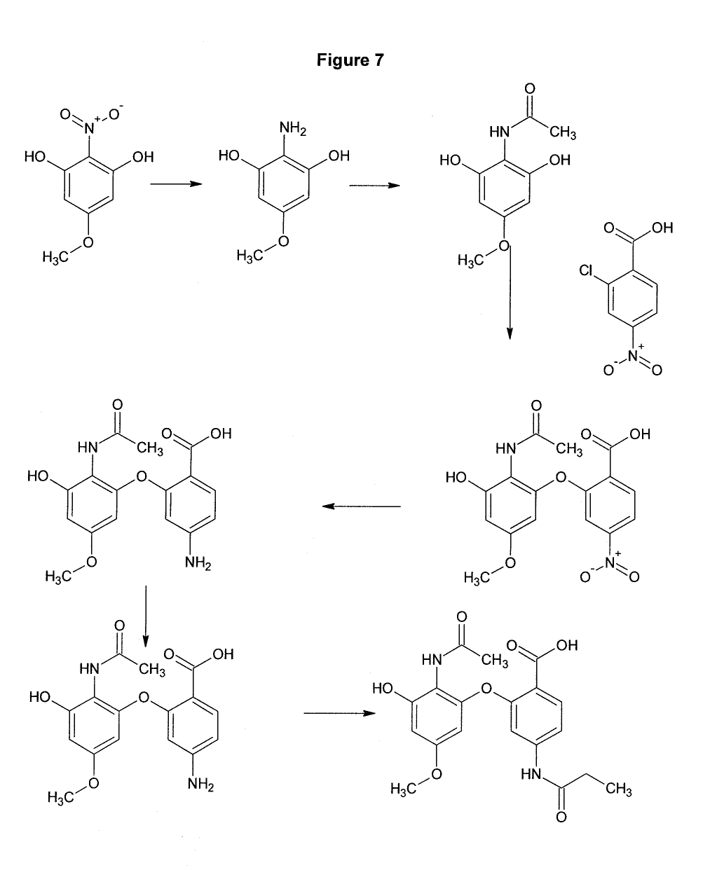

FIG. 7 Chemical synthesis of compound 1F (d408)

FIG. 8 Comparison of the structure of the hyaluronan disaccharide and compound 1D

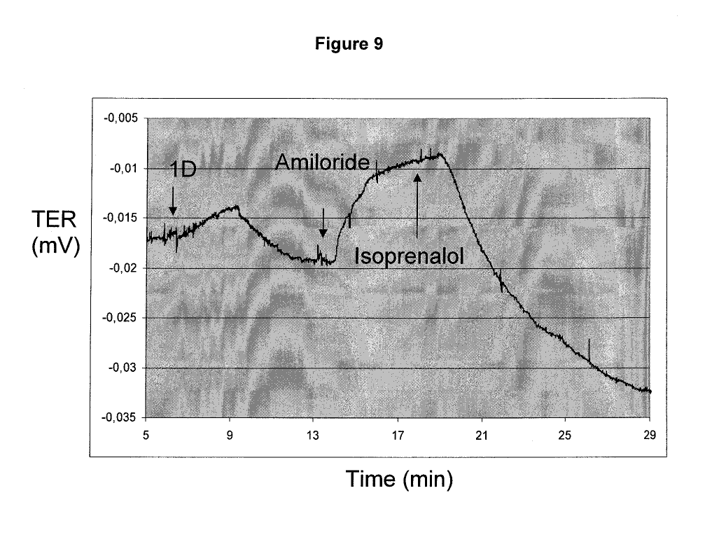

FIG. 9 Effect of compound 1D on the transepithelial resistance

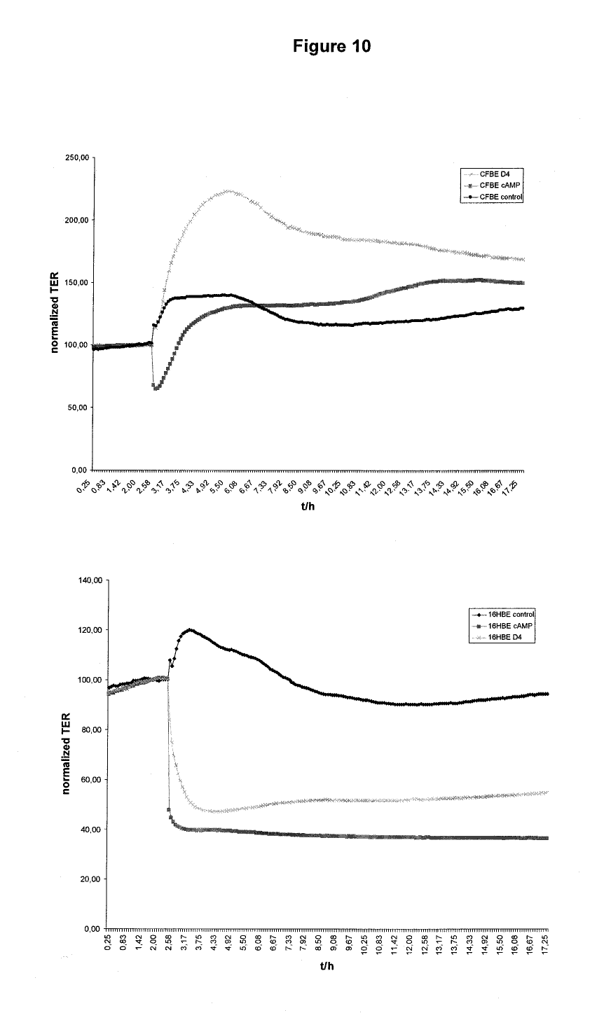

FIG. 10 This shows the effect of D4 (which is identical with compound 1D as defined herein—it will be understood that the compound 1D of the present invention is identical to compound D4 which is partially mentioned in the examples and figures) on the transepithelial resistance of epithelial cells containing normal and F508-CFTR in comparison the CFTR-activator 8-Bromo-cAMP. In normal cells the resistance drops immediately in both cases. In F508-cells the resistance increases as compared to 8-Bromo-cAMP and control.

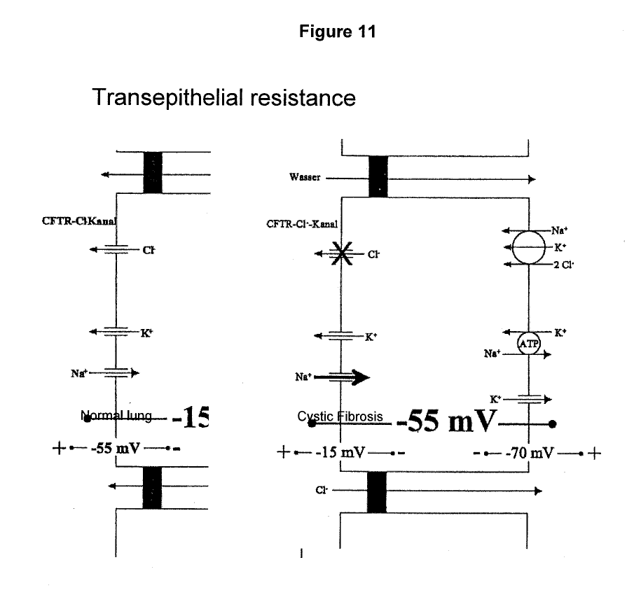

FIG. 11 This figure explains the above observation by the different behaviour of normal and cystic fibrosis epithelial cells. In normal cells, activation of CFTR further reduces the TER. In F508 there is no chloride efflux via CFTR and a massive Na+ influx that is responsible for most of apical membrane current. The transcellular potential (=resistance) is much higher than in normal cells. Opening the existing CFTRchannels reduces the Na+ influx and thus increases even more the resistance. It is also seen that the TER peaks at about 5 hours and than gradually decreases.

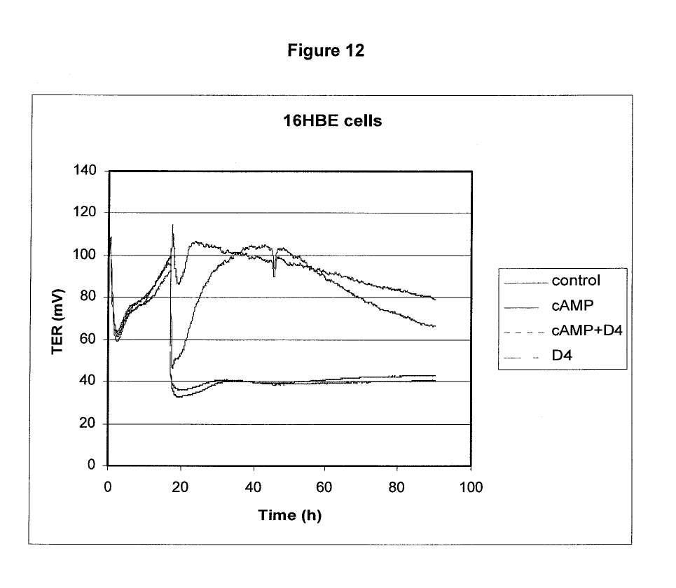

FIG. 12 These figures show the TER over a period of 90 hours. In normal cells the CFTR channel remain open and reduce the TER. In F508-CFTR, the resistance drops below the control without any treatment. This effect is probably due to recruitment of novel functionally intact by transcription and translation. Therefore, D4 has dual effects. It immediately opens existing CFTR, and the long term effect of is a permanent recovery of functionally active CFTR. This phenomenon is called recovery of rescue.

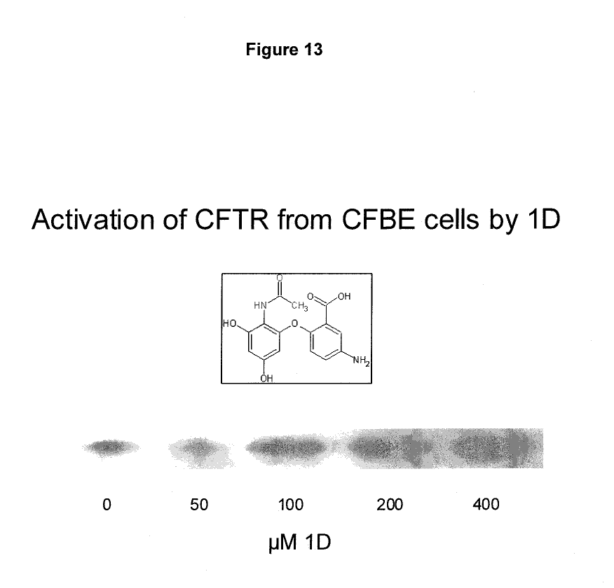

FIG. 13 This is a Western blot of CFTR from surface of epithelial cells with defective F508-CFTR exposed to 1D in a concentration dependent manner. It verifies that the CFTR is indeed recruited to the plasma membrane upon addition of 1D.

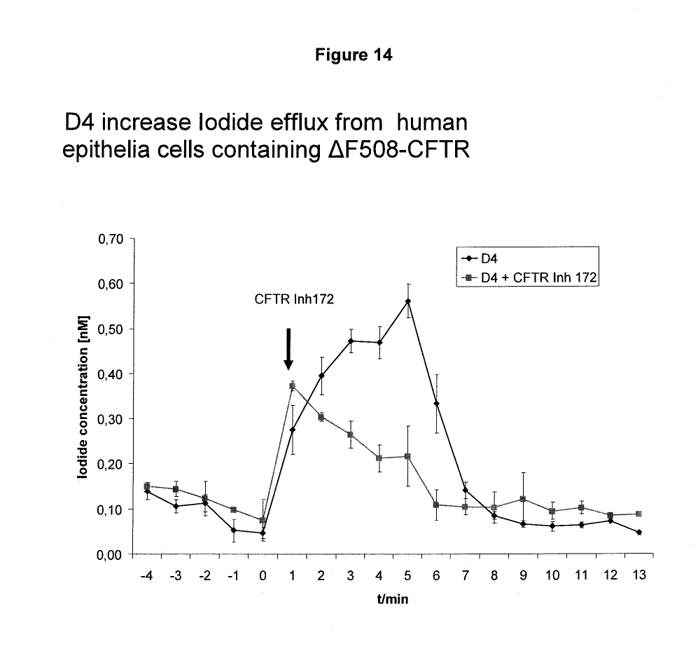

FIG. 14 CFTR can also export iodide instead of chloride. We made use of this property to measure the kinetics of export with an iodide sensitive electrode. This figure shows that upon addition of D4 (blue) at the time indicated, defective F508-CFTR channels immediately open. Simultaneous addition of the CFTR-specific inhibitor CFTR172 reduces the activation. (D4 corresponds to compound 1D)

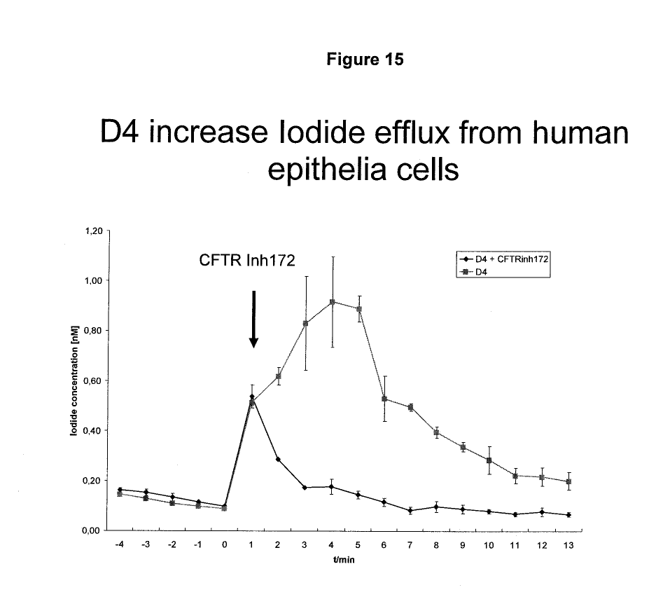

FIG. 15 The same effect as depicted in FIG. 14 is observed with cells containing normal CFTR.

### EXAMPLES

The following examples illustrate the invention. These examples should not be construed as to limit the scope of this invention. The examples are included for purposes of illustration and the present invention is limited only by the claims.

Human epithelial cells containing wildtype CFTR (16HBE14o-) and the mutant cell line CFBE14o- were kindly provided by Dr. D. C. Gruenert [22]. They were grown in suspension culture in Dulbeccós medium supplemented with streptomycin/penicillin (100 units of each/ml) and 10% foetal calf serum.

The cytotoxicity of the drugs was measured as described [23].

### Example 1

### Chemical Synthesis of Compound 1D

Nitrophloroglucinol (1 g, 6.5 mMol) was dissolved in 10 ml of methanol and hydrogenated in a hydrogen atmosphere in the presence of 0.1 g of 10% Pd/C overnight at room temperature. The solvent was removed by evaporation an the residue was dissolved in 12 ml of dimethylformamide. 2-chlor-5-nitrobenzoic acid (1.2 g; 6 mMol), 1.7 g of K2CO3, 0.18 g of copper powder and 0.18 g of CuCl were added and the mixture was refluxed for 3 hours. After cooling to room temperature, 12 ml of concentrated HCl and 120 ml of water were added, and the product was extracted with 120 of ethylacetate. The organic phase was dried over Na2SO4 and evaporated. The product was dissolved in 12 ml of methanol; 0.1 g of palladium (10% ob charcoal) was added and hydrogenated in a hydrogen atmosphere overnight at room temperature. The catalyst was removed by centrifugation, and the solvant was evaporated to obtain compound 1D.

### Example 1a

### Chemical Synthesis of Compound 1B

Compound 1B was prepared by the same procedure substituting 2-chloro-5-nitrobenzoic acid with 2-chloro-benzoic acid and omitting the second catalytical hydrogenation.

### Example 1b

### Chemical Synthesis of Compound 1C

Compound 1C was prepared by the same procedure substituting nitrophloroglucinol with 2-chloro-nitrobenzene and 2-chlor-5-nitrobenzoic acid with 2,6-dihydroxy-benzoic acid and omitting the first catalytical hydrogenation.

### Example 2

### Transepithelial Resistance

A cell monolayer on a thin filter membrane (growth area, 4.2 cm2; pore diameter, 0.4 μm; thickness, 20 μm; Falcon, Heidelberg, Germany) served as a test barrier for the invasive capabilities of malignant cells. When confluent, these cells from a tight epithelial sheet with a high trans-epithelial electrical resistance (TEER), which was measured continuously using a STX-2 electrode (WPI, Sarasota, USA). Permeabilization of the epithelial cell layer (MDCK-C7 cells) due to the invasive activity of the melanoma cells can be determined by trans-epithelial electrical resistance (TEER) measurements, as previously reported [24;25].

### Example 3

### Determination of Hyaluronan Export

The cells were incubated for 24 hours at 37° C., the media were replaced with fresh media and after additional 24 hours aliquots (5 and 20 μl) of the culture medium were used for measurement of the hyaluronan concentration in the cell culture medium by an ELISA. The wells of a 96 well Covalink-NH-microtiter plate (NUNC) were coated with 100 μl of a mixture of 100 mg/ml of hyaluronan (Healon®), 9.2 μg/ml of N-Hydroxysuccin-imide-3-sulfonic acid and 615 μl/ml of 1-ethyl-3-(3-dimethylaminopropyl)-carbodiimide for 2 hours at room temperature and overnight at 4° C. The wells were washed three times with 2 M NaCl, 41 mM MgSO4, 0.05% Tween-20 in 50 mM phosphate buffered saline pH 7.2 (buffer A) and once with 2 M NaCl, 41 mM MgSO4, in phosphate buffered saline pH 7.2. Additional binding sites were blocked by incubation with 300 μl of 0.5% bovine serum albumin in phosphate buffered saline for 30 min at 37° C. Calibration of the assay was performed with standard concentrations of hyaluronan ranging from 15 ng/ml to 6000 ng/ml in equal volumes of culture medium as used for measurement of the cellular supernatants. A solution (50 μl) of the biotinylated hyaluronan binding fragment of aggrecan (Applied Bioligands Corporation, Winnipeg, Canada) in 1.5 M NaCl, 0.3 M guanidinium hydrochloride, 0.08% bovine serum albumin 0.02% NaN3 25 mM phosphate buffer pH 7.0 was preincubated with 50 μl of the standard hyaluronan solutions or cellular supernatants for 1 hour at 37° C. The mixtures were transferred to the hyaluronan-coated test plate and incubated for 1 hour at 37° C. The microtiter plate was washed three times with buffer A and incubated with 100 μl/well of a solution of streptavidin-horseraddish-peroxidase (Amersham) at a dilution of 1:100 in phosphate buffered saline, 0.1% Tween-20 for 30 min at room temperature. The plate was washed five times with buffer A and the colour was developed by incubation with a 100 μl/well of a solution of 5 mg o-phenylenediamine and 5 μl 30% H2O2 in 10 ml of 0.1 M citrate-phosphate buffer pH 5.3 for 25 min at room temperature. The adsorption was read at 490 nm. The concentrations in the samples were calculated from a logarithmic regression curve of the hyaluronan standard solutions.

### Example 4

### Iodide Efflux

Iodide efflux experiments were performed as described [26]. Briefly, Cells (80-90% confluent) were incubated for 1 h in a loading buffer containing 136 mM NaI, 3 mM KNO3, 2 mM Ca(NO3)2, 11 mM glucose, and 20 mM Hepes, adjusted to pH 7.4 with NaOH. To remove extracellular iodide, cells were thoroughly washed with efflux buffer (136 mM NaNO3 replacing 136 mM NaI in the loading buffer) and then equilibrated in 2.5 ml efflux buffer for 1 min. The efflux buffer was changed at 1 min intervals over the duration of the experiment. Four minutes after anion substitution, cells were exposed to compound 1D. The amount of iodide in each 2.5 ml sample of efflux buffer was determined using an iodide-selective electrode (HNU Systems Ltd, Warrington, UK). Cells were loaded and experiments performed at room temperature.

### Example 5

### Western Blotting of Cell Surface Expressed CFTR

The cell pellets were solubilized by vortexing in buffer (Tris-HCl 0.06 M; 2% SDS, 10% glycerol, 0.1M dithiothreitol, 0.1% bromophenol-blue and the protease inhibitor cocktail, pH 6.8). Following centrifugation (2 min, 8.000 g) samples of the supernatant were separated on 10% poly-acrylamide slabgels. Proteins were subsequently electroblotted onto nitrocellulose paper in 0.025 M Tris, 0.192 M glycine, 20% methanol. The blots were incubated at 4° C. with 0.02M Tris-HCl, 0.15M NaCl, 0.1% Tween20, pH7.5 followed by overnight incubation at 4° C. with a 1:500 dilution of primary anti-CFTR antibody in 0.02M Tris-HCl, 0.15M NaCl, 0.1% Tween20, pH7.5. Blots were washed three times, incubated with peroxidase-conjugated anti-rabbit IgG for 2 h, and washed four times. Peroxidase activity was detected with bioluminescence reagent (ECL kit; Amersham, Braunschweig, Germany) on X-ray film.

### Example 6

### Chemical Synthesis of Compound 1F (d408)

Nitration of 5-methoxyresorcinol

5-Methoxyresorcinol (5 g) was dissolved in 70 ml of a 1:1 mixture of sulfuric acid and water. HNO3 (4.73 ml) was mixed with 22.5 ml of a 1:1 mixture of sulfuric acid and water and dropped slowly to the stirring solution of 5-methoxyresorcinol holding the temperature below 20° C. The mixture was stirred for 1 hour and poured onto 90 g of ice. The precipitate of Nitro-5-methoxyresorcinol was filtered off and washed with cold water.

### Hydrogenation and Acetylation

Nitro-5-methoxyresorcinol was dissolved in ethylacetate. The hydrogenation catalyst 10% paladium on charcoal was added and the solution was stirred under a ballon pressure of hydrogen at room temperature overnight. The solution was filtered and evaporated. The residue was dissolved in 20 ml of an aqueous solution of NaHCO3 and acetic anhydride was added dropwise. The solution was stirred overnight and extracted with ethylacetate. Te organic layer was dried with Na2SO4 and evaporated.

### Coupling Reaction

Acetamino-5-methoxyresorcinol (3.5 g), K2CO3 (7.5 g), Cu (0.15 g), CuCl2 (0.15 g) were suspended in 150 of dimethylformamide and refluxed under an atmosphere of nitrogen. A solution of 2-chloro-4-nitrobenzoic acid (3.0 g) in 30 ml of dimethylformamide was added dropwise. The mixture was refluxed for an additional hour, and cooled to room temperature. Undissolved material was removed by centrifugation and the solution was mixed with 500 ml of cold dilute HCl. The water phase was extracted with ethylacetate, and organic phase was dried and evaporated.

### Hydrogenation and Propionylation

The product of the above coupling reaction was dissolved in ethylacetate and hydrogenated as described above. The resulting amine was reacted with 2.7 ml of propionylchloride. The propionylated product was extracted with ethylacetate, the organic layer was dried and evaporated for form a crystalline product. The purity was confirmed by thin layer chromatrography with a mixture of chloroform and methanol (9:1).

### Example 7

### Effect of Compound 1D on the Transepithelial Nasal Resistance

The effect of compound 1D was evaluated on the inventor of the present application by measurement of the transepithelial nasal resistance. This is a standard protocol for testing pharmaceutically active compounds on humans [1], (http://central.igc.gulbenkian.pt/cftr/vr/e/schuler_basic_protocol_for_measurement_of_transepithelial_nasal_potential_difference.pdf) (see also the results of FIG. 9).

The nasal epithelium was equilibrated with isotonic (0.9%) NaCl, 2 mM CaCl2. After a baseline was reached at about 5 min, the solution was changed to isotonic NaCl, 2 mM CaCl2 containing 100 μM 1D. After a transient increase in the resistance, the resistance decreased below the equilibrating solution indicating that the chloride channels had opened. To determine the maximal possible potential differences in this experiment, the solution was changed to isotonic NaCl, 2 mM CaCl2 containing 10 μM amiloride that is known to close Na+ channels. The resistance increased to a maximal valued. After equilibration, the solution was changed to low salt with 0.09% NaCl, 0.2 mM CaCl2 containing 10 μM isoprenalol. This caused all channels to opened and a maximal drop of the transepithelial resistance.

Since it is known that the CFTR conduction constitutes only 15% of the total transepithelial ion flow, the extent of the conductivity drop in FIG. 9 suggests that it acted specifically on CFTR. In addition, the data indicated that the effect of 1D was reversible, because it could be washed out.
[1] Schuler, D., Sermet-Gaudelus, I., Wilschanski, M., Ballmann, M., Dechaux, M., Edelman, A., Hug, M., Leal, T., Lebacq, J., Lebecque, P., Lenoir, G., Stanke, F., Wallemacq, P., Tummler, B., and Knowles, M. R. (2004) Basic protocol for transepithelial nasal potential difference measurements. J Cyst. Fibros. 3 Suppl 2, 151-155.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, detailed Description, and Examples is hereby incorporated herein by reference.

### REFERENCES

[1] Bobadilla, J. L., Macek, M., Jr., Fine, J. P., & Farrell, P. M. (2002) Cystic fibrosis: a worldwide analysis of CFTR mutations—correlation with incidence data and application to screening. Hum. Mutat., 19, 575-606.
[2] Pilewski, J. M. & Frizzell, R. A. (1999) Role of CFTR in airway disease. Physiol Rev., 79, S215-S255.
[3] Sheppard, D. N. & Welsh, M. J. (1999) Structure and function of the CFTR chloride channel. Physiol Rev., 79, S23-S45.
[4] Denning, G. M., Anderson, M. P., Amara, J. F., Marshall, J., Smith, A. E., & Welsh, M. J. (1992) Processing of mutant cystic fibrosis transmembrane conductance regulator is temperature-sensitive. Nature, 358, 761-764.
[5] Lukacs, G. L., Mohamed, A., Kartner, N., Chang, X. B., Riordan, J. R., & Grinstein, S. (1994) Conformational maturation of CFTR but not its mutant counterpart (deltaF508) occurs in the endoplasmic reticulum and requires ATP. EMBO J, 13, 6076-6086.
[6] Du, K., Sharma, M., & Lukacs, G. L. (2005) The DeltaF508 cystic fibrosis mutation impairs domain-domain interactions and arrests post-translational folding of CFTR. Nat. Struct. Mol. Biol., 12, 17-25.
[7] Sato, S., Ward, C. L., Krouse, M. E., Wine, J. J., & Kopito, R. R. (1996) Glycerol reverses the misfolding phenotype of the most common cystic fibrosis mutation. J. Biol. Chem., 271, 635-638.
[8] Rubenstein, R. C., Egan, M. E., & Zeitlin, P. L. (1997) In vitro pharmacologic restoration of CFTR-mediated chloride transport with sodium 4-phenylbutyrate in cystic fibrosis epithelial cells containing deltaF508-CFTR. J. Clin. Invest, 100, 2457-2465.
[9] Dalemans, W., Barbry, P., Champigny, G., Jallat, S., Dott, K., Dreyer, D., Crystal, R. G., Pavirani, A., Lecocq, J. P., & Lazdunski, M. (1991) Altered chloride ion channel kinetics associated with the deltaF508 cystic fibrosis mutation. Nature, 354, 526-528.
[10] Haws, C. M., Nepomuceno, I. B., Krouse, M. E., Wakelee, H., Law, T., Xia, Y., Nguyen, H., & Wine, J. J. (1996) DeltaF508-CFTR channels: kinetics, activation by forskolin, and potentiation by xanthines. Am. J. Physiol, 270, C1544-C1555.
[11] Drumm, M. L., Wilkinson, D. J., Smit, L. S., Worrell, R. T., Strong, T. V., Frizzell, R. A., Dawson, D. C., & Collins, F. S. (1991) Chloride conductance expressed by deltaF508 and other mutant CFTRs in Xenopus oocytes. Science, 254, 1797-1799.
[12] Hwang, T. C., Wang, F., Yang, I. C., & Reenstra, W. W. (1997) Genistein potentiates wild-type and deltaF508-CFTR channel activity. Am. J. Physiol, 273, C988-C998.
[13] Al-Nakkash, L. & Hwang, T. C. (1999) Activation of wild-type and deltaF508-CFTR by phosphodiesterase inhibitors through cAMP-dependent and -independent mechanisms. Pflugers Arch., 437, 553-561.
[14] Yoo, C. L., Yu, G. J., Yang, B., Robins, L. I., Verkman, A. S., & Kurth, M. J. (2008) 4′-Methyl-4,5′-bithiazole-based correctors of defective deltaF508-CFTR cellular processing. Bioorg. Med. Chem. Lett., 18, 2610-2614.
[15] Loo, T. W., Bartlett, M. C., & Clarke, D. M. (2008) Correctors promote folding of CFTR in the endoplasmic reticulum. Biochem. J., . . . .
[16] Yoo, C. L., Yu, G. J., Yang, B., Robins, L. I., Verkman, A. S., & Kurth, M. J. (2008) 4′-Methyl-4,5′-bithiazole-based correctors of defective DeltaF508-CFTR cellular processing. Bioorg. Med. Chem. Lett., . . . .
[17] Wang, Y., Loo, T. W., Bartlett, M. C., & Clarke, D. M. (2007) Correctors promote maturation of cystic fibrosis transmembrane conductance regulator (CFTR)-processing mutants by binding to the protein. J. Biol. Chem., 282, 33247-33251.
[18] Carlile, G. W., Robert, R., Zhang, D., Teske, K. A., Luo, Y., Hanrahan, J. W., & Thomas, D. Y. (2007) Correctors of protein trafficking defects identified by a novel high-throughput screening assay. Chembiochem., 8, 1012-1020.
[19] Pedemonte, N., Lukacs, G. L., Du, K., Caci, E., Zegarra-Moran, O., Galietta, L. J., & Verkman, A. S. (2005) Small-molecule correctors of defective DeltaF508-CFTR cellular processing identified by high-throughput screening. J. Clin. Invest, 115, 2564-2571.
[20] Yang, H., Shelat, A. A., Guy, R. K., Gopinath, V. S., Ma, T., Du, K., Lukacs, G. L., Taddei, A., Folli, C., Pedemonte, N., Galietta, L. J., & Verkman, A. S. (2003) Nanomolar affinity small molecule correctors of defective DeltaF508-CFTR chloride channel gating. J. Biol. Chem., 278, 35079-35085.
[21] Schulz, T., Schumacher, U., Prante, C., Sextro, W., & Prehm, P. (2008) Hyaluronan export by CFTR is defective in patients with cystic fibrosis. submitted.
[22] Kunzelmann, K., Schwiebert, E. M., Zeitlin, P. L., Kuo, W. L., Stanton, B. A., & Gruenert, D. C. (1993) An immortalized cystic fibrosis tracheal epithelial cell line homozygous for the deltaF508 CFTR mutation. Am. J. Respir. Cell Mol. Biol., 8, 522-529.
[23] O'Brien, J., Wilson, I., Orton, T., & Pognan, F. (2000) Investigation of the Alamar Blue (resazurin) fluorescent dye for the assessment of mammalian cell cytotoxicity. Eur. J. Biochem., 267, 5421-5426.
[24] Ludwig, T., Ossig, R., Graessel, S., Wilhelmi, M., Oberleithner, H., & Schneider, S. W. (2002) The electrical resistance breakdown assay determines the role of proteinases in tumor cell invasion. Am. J. Physiol Renal Physiol, 283, F319-F327.
[25] Schnaeker, E. M., Ossig, R., Ludwig, T., Dreier, R., Oberleithner, H., Wilhelmi, M., & Schneider, S. W. (2004) Microtubule-dependent matrix metalloproteinase-2/matrix metalloproteinase-9 exocytosis: prerequisite in human melanoma cell invasion. Cancer Res., 64, 8924-8931.
[26] Lansdell, K. A., Kidd, J. F., Delaney, S. J., Wainwright, B. J., & Sheppard, D. N. (1998) Regulation of murine cystic fibrosis transmembrane conductance regulator Cl-channels expressed in Chinese hamster ovary cells. J Physiol, 512 (Pt 3), 751-764.
[27] Mall, M., Bleich, M., Greger, R., Schreiber, R., & Kunzelmann, K. (1998) The amiloride-inhibitable Na+ conductance is reduced by the cystic fibrosis transmembrane conductance regulator in normal but not in cystic fibrosis airways. J. Clin. Invest, 102, 15-21.
[28] Bachhuber, T., Konig, J., Voelcker, T., Murle, B., Schreiber, R., & Kunzelmann, K. (2005) Cl-interference with the epithelial Na+ channel ENaC. J. Biol. Chem., 280, 31587-31594.
[29] Kopito, R. R. (1999) Biosynthesis and degradation of CFTR. Physiol Rev., 79, S167-S173.
[30] Gentzsch, M., Chang, X. B., Cui, L., Wu, Y., Ozols, V. V., Choudhury, A., Pagano, R. E., & Riordan, J. R. (2004) Endocytic trafficking routes of wild type and DeltaF508 cystic fibrosis transmembrane conductance regulator. Mol. Biol. Cell, 15, 2684-2696.
[31] Johnson, L. G., Olsen, J. C., Sarkadi, B., Moore, K. L., Swanstrom, R., & Boucher, R. C. (1992) Efficiency of gene transfer for restoration of normal airway epithelial function in cystic fibrosis. Nat. Genet., 2, 21-25.

1. A compound which is characterized by the formula 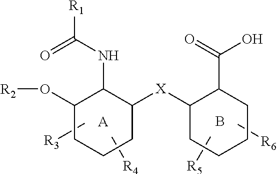
or a pharmaceutically acceptable salt, solvate, hydrate thereof,
wherein
the ring systems A and B are independently selected from a monosaccharide, aryl (preferably phenyl), a heteroaryl or cycloalkyl (preferably cyclohexan),
preferably with all substituents in equatorial configurations;
R1 is independently selected from alkyl (preferably C1 to C6), a substituted or unsubstituted phenyl, preferably CH3;
R2 is H, alkyl (preferably C1 to C6), a carbohydrate in a glycosidic β-linkage, preferably H;
R3, R4, R5, and R6 are independently selected from H, (OH) hydroxy, alkyl preferably C1 to C6, alkoxy (preferably C1 to C6), amino, alkylamino (preferably C1 to C6), halogen, benzylamino, or benzoylamino;
X is O, NH, alkylamino (NR), CO, S; and
for use in the treatment of (for treating) and/or preventing a disease or medical condition which is associated with mutant cystic fibrosis transmembrane conductance regulator (CFTR).

2. The compound of claim 1 wherein said disease or medical condition which is associated with mutant CFTR cystic fibrosis (CF).

3. A method for manufacturing a pharmaceutical composition comprising the steps of formulating the compound defined in claim 1 in a pharmaceutically acceptable form.
